(19) United States
(12) Patent Application Publication
(10) Pub. No.: US 2021/0212923 A1
(43) Pub. Date: Jul. 15, 2021

(54) COSMETIC COMPOSITION COMPRISING ROSE EXTRACTS

(71) Applicant: L V M H Recherche, Saint Jean de Braye (FR)

(72) Inventors: Marc Dumas, Saint Jean Le Blanc (FR); Jocelyne Franchi, Saint Jean de la Ruelle (FR); Valentin Jeronimo, Trainou (FR); Virginie Pecher, La Chapelle Saint Mesmin (FR); Kristell Lazou, Orleans (FR)

(73) Assignee: L V M H Recherche, Saint Jean de Braye (FR)

(21) Appl. No.: 16/613,953

(22) PCT Filed: May 16, 2018
(86) PCT No.: PCT/EP2018/062775

§ 371 Date: Nov. 15, 2019

(30) Foreign Application Priority Data
May 16, 2017 (FR) 1754304

(51) Int. Cl. A61K 8/9789; A61Q 19/08; A61K 8/06

(52) U.S. Cl. CPC A61K 8/9789; A61Q 19/08; A61K 8/06; A61K 2800/524; A61K 2800/21; A61K 2800/56; A61K 2800/522; A61K 2800/10

(57) ABSTRACT

The present invention relates to a cosmetic composition for topical application to the skin comprising, in a physiologically acceptable medium, at least an effective amount of at least one aqueous extract of rose and of at least one oily extract of rose, and the use thereof in particular for promoting the natural rhythmic process of skin cells and/or improving the micro-nutritional balance of the skin.

### FIELD OF THE INVENTION

The present invention relates to a cosmetic composition comprising at least one aqueous extract of rose and one oil extract of rose, and their use in particular to promote the natural rhythmic process of skin cells and/or to improve the micronutritional balance of the skin.

### STATE OF THE ART

It is well known that the quality of nutrition is reflected in the health of the skin and that certain skin disorders are associated with deficiencies, particularly in certain micronutrients (Park K. Role of micronutrients in skin health and function. Biomol. Ther. 2015; 23; 207-217).

The micronutritional balance of the skin can be affected by external and/or internal factors, such as fatigue, stress, oxidative effects, UV exposure, and/or cell senescence.

In particular, subjects exposed to such factors are observed to have:
a lower level of hydration (this difference increases with age);
a deficit in essential fatty acids (omega 3 and omega 6);
a deficit in long-chain fatty acids in ceramides (poorer barrier function); and/or
a lower squalene content (alteration of the hydrolipidic film protecting the skin surface).

These observations highlight imbalances, particularly with regard to micronutrients.

It is known that these nutrients, or nutritional components, contribute at the cutaneous level in particular to the establishment of an environment favourable to barrier function repair, to skin hydration and thus, in the long term, to rejuvenated skin that is less vulnerable to premature ageing.

Micronutrients are essential for the development and formation of the skin (an organ in constant evolution), for the physiological renewal of the epidermis and for the adaptation of the skin to its environment (as protective envelope). They each have a particular role, complementary functions that cover the many facets of skin metabolism (Park K. Role of micronutrients in skin health and function. Biomol. Ther. 2015, 23:207-217; Polefka T. Interaction of mineral salts with the skin: a literature survey. Int J. Cosmetic. Sci. 2012, 34:416-423; Boelsma E. Nutritiaonal skin care: health effects of micronutrients and fatty acids. Am. J. Clin. Nutr. 2001, 73:853-864; Winkler P. Minerals and the skin in Nutrition and the skin-Lessons for anti-ageing, beauty and healthy skin. 2011, Apostolos Papas Editor; Chap 7: 91-109), and mainly:
participate in antioxidant protection (vitamins A, E, vitamin C, zinc, selenium);
are essential for lipid, carbohydrate or protein metabolism (vitamins B3, B5, vitamin C for collagen synthesis), energy production (vitamin B2, magnesium);
contribute to the proper functioning of enzymes, by providing the metal ions necessary for their activity (copper, manganese, selenium, zinc, iron);
regulate epidermal differentiation (calcium);
are involved in the maintenance of membrane potential, in fluid balance, and have a role in skin hydration (potassium and sodium);
contribute to the strengthening of the hydrolipidic film that helps the skin to maintain its elasticity and suppleness (essential fatty acids):
linoleic acid (omega 6) is involved in the manufacture of cell membranes, and is used in the composition of ceramides;
   alpha-linolenic acid (omega 3) is also involved in membrane fluidity and these eicosapentaenoic acid (EPA) and docosahexaenoic acid (DHA) derivatives are known for their anti-inflammatory properties;
   oleic acid (omega 9, non-essential) is known for its nourishing, repairing and healing properties.

The use of vitamins (A, E, C) and/or essential fatty acids is known in the formulation of cosmetic compositions for the skin, in particular for their protective and/or nutritional effect, but there is still a need for new compounds to maintain and/or promote the nutritional balance of the skin and therefore stimulate hydration, the skin barrier, and/or skin regeneration.

However, it has been demonstrated that many metabolic pathways are under the control of clock genes, present in each cell of the body.

Skin cells also have such clock genes that function in a circadian and autonomous manner, thus constituting a functional cellular biological clock (Sandu C. Human skin keratinocytes, melanocytes, and fibroblasts contain distinct circadian clock machineries Cell. Mol. Life Sci. 2012, 19:3329-3339). These clock genes control the rate and intensity of expression of genes involved in the quality of the skin surface and barrier, in hydration, in resistance and/or regeneration of the skin.

Analysis of the rhythmic expression of clock genes in epidermal keratinocytes makes it possible to subdivide the 24 hours of a day into 5 successive periods. Each period corresponds to the expression of a cohort of genes involved in different metabolic pathways; more than 5000 genes are under their control. These metabolic pathways include that of lipid metabolism (ceramide synthesis, fatty acid transport, etc.), that of glucose metabolism and calcium homeostasis: synthesis and organization of skin barrier lipids, glucose utilization, epidermal maturation under calcium control, all important in skin nutrition. It has been shown that these metabolic pathways are under the control of 4 clock genes Period 1 (PER1), Period 2 (PER2), Period 3 (PER3) and Cryptochrome Circadian Clock 2 (CRY2) (Janich P., Human epidermal stem cell function is regulated by circadian oscillations. Cell Stem Cell 2013, 13, 1-9).

Unexpectedly, the Applicant has shown the in vitro effect of an aqueous extract of rose and the effect of an oil extract of rose on stimulating the expression of several clock genes on normal human epidermal keratinocytes in culture, notably with complementary effects: the aqueous extract of rose according to the invention plays a role in the expression of the CRY2, PER1 and PER3 genes while the oil extract of rose according to the invention plays a role in the expression of the PER2 gene. The use of an aqueous extract of rose and an oil extract of rose is therefore advantageous to maintain and/or stimulate the natural rhythmic process of the skin cells, essential to the above-mentioned metabolic pathways.

In parallel, the Applicant has also observed effects of these extracts on the target genes of these clock genes involved in particular in lipid metabolism and barrier function: thus, the oil extract of rose (‘satin oil’) stimulates in human keratinocytes the expression of ceramide synthase (CERS3) involved in lipid metabolism, calmodulin 3 (CALM3) which binds calcium and participates in particular in the regulation of the cell cycle, keratin 1 (KRT1) involved in cell differentiation, gap junction alpha-1 protein (GjA1) also known as connexin 43 (Cx43) involved in cell communication and transport and desmocollin 3 (DSC3) involved in cell cohesion; the aqueous extract of rose (‘rose cryoextract’), in turn, stimulates in human keratinocytes the protein expression of keratin 10 (KRT10), a marker of epidermal maturation, and desmoglein 1 (DSG1), a marker of epidermal cohesion.

Preferably, these two rose extracts are used according to the invention in a formulation that allows their full potential to be expressed: thus, each rose extract, depending on its polarity, is used respectively in the aqueous phase for the aqueous extract and in the fat or oil phase for the oil extract, thus allowing them a better dispersion for an optimized efficiency after application on the skin.

### SUMMARY OF THE INVENTION

A first subject-matter of the invention relates to a cosmetic composition for topical application to the skin comprising, in a physiologically acceptable medium, at least an effective amount of at least one aqueous extract of rose and at least one oil extract of rose. According to a particular embodiment, the aqueous extract of rose and the oil extract of rose are extracts of rose flowers.

Another subject-matter of the invention relates to a cosmetic process to promote the natural rhythmic process of skin cells and/or the micronutritional balance of the skin, comprising the application to the skin, in particular of the body, face and/or neck and in particular of the face and/or neck, of a cosmetic composition as defined according to the invention.

According to the invention, ‘natural rhythmic process of skin cells’ means in particular the natural process involving the clock genes of skin cells that function in a circadian and autonomous manner, whose role is to control the rate and intensity of expression of target genes involved in many metabolic pathways at the level of skin cells.

According to the invention, ‘micronutritional balance of the skin’ means in particular the presence of micronutrients naturally present in the skin, in balanced quantity and diversity, provided in particular by food.

The invention also relates to the non-therapeutic cosmetic use of at least an effective amount of at least one aqueous extract of rose and at least one oil extract of rose in a cosmetic composition, as an agent to promote and/or improve the natural rhythmic process of skin cells, the micronutritional balance of the skin, the lipid metabolism of the skin, the skin barrier function, the hydration and/or regeneration of the skin.

In particular, the effective amount of at least one aqueous extract of rose and at least one oil extract of rose stimulates the expression of epidermal clock genes and the expression of epidermal clock target genes, involved in particular in lipid metabolism, the skin barrier, cell differentiation, cell communication and/or cell cohesion.

### DETAILED DESCRIPTION OF THE INVENTION

The invention therefore relates to a cosmetic composition for topical application to the skin comprising, in a physiologically acceptable medium, at least an effective amount of at least one aqueous extract of rose and at least one oil extract of rose.

The aqueous extract of rose and the oil extract of rose are present in the cosmetic composition of the invention in an amount effective to obtain the desired effect.

In particular, they will be respectively present in contents ranging from 0.1% to 10%, in particular from 0.5% to 5%, or even from 1% to 4% by weight of raw material based on the total weight of the composition. Illustrative examples are given below.

According to a particular and preferred embodiment of the invention, the cosmetic composition of the invention comprises at least one aqueous phase comprising said aqueous extract of rose and at least one fat or oil phase comprising said oil extract of rose.

### Plant Material

The extracts of the invention are rose extracts. According to the invention, the terms extract of rose, extract of rose bush and extract of the genus Rosa are used interchangeably.

This extract can be chosen from the different parts of the plant or mixtures thereof, in particular a leaf extract, a flower (petal) extract, a sepal extract, a wood (stem) extract or a mixture thereof.

Advantageously, it will be a flower extract, and in particular an extract of fresh flowers.

The genus Rosa includes more than 1000 species, including Rosa damascena, Rosa multiflora, Rosa centifolia, Rosa rugosa, Rosa chinensis, Rosa moschata, Rosa alba, Rosa alpina, Rosa canina, Rosa cinnamonea, Rosa gallica, Rosa repens, Rosa rubrifolia, Rosa rubiginosa, Rosa sempervirens, Rosa spinosissima, Rosa styosa, Rosa tomentosa and Rosa villosa.

The skilled person will preferably choose roses selected with disease resistance, which are vigorous and flowering, in particular with pastel shades and whose properties are preserved by an organic environment and farming method. According to a particular and preferred embodiment, an extract of rose flowers of the Evanrat or “Jardin de Granville®” rose variety will be used.

The “Jardin de Granville®” rose bush or rose is a hybrid variety offered exclusively by “Roses anciennes André Eve S.A.S” and protected by plant breeder's rights under No 20110345 with the name Rosa L. as species and the name EVANRAT for the variety. This rose bush belongs to the group of modern hybrids, which, from May to October, is permanently covered with roses, thus showing an excellent flowering character. In particular, it is found under the INCI name ‘Rosa Hybrid Flower Extract’.

Thus, according to a particular and preferred embodiment of the invention, the cosmetic composition according to the invention is characterized in that the aqueous extract of rose and the oil extract of rose are extracts of rose flower, preferably extracts of rose flower of the Evanrat or Jardin de Granville® rose variety.

The rose extracts according to the invention are extracts concentrated in natural (non-volatile) compounds, respectively in polar natural compounds for the aqueous extract and in non-polar natural compounds for the oil extract. The extracts according to the invention are distinct from rose essential oils or rose waters that contain volatile compounds. A rose essential oil is obtained by extracting or distilling volatile molecules from rose (e.g. terpenoids, aromatic molecules) and a rose water or rose hydrosol is obtained by distilling rose petals and contains volatile perfuming molecules.

According to the invention, they are extracts in particular of rose flowers implementing the extraction of natural (non-volatile) compounds from rose flowers in the presence of solvents, respectively aqueous (polar) or oil (non-polar), and the use, in the formulation, in the form of an aqueous or oil solution or concentrate, the solvent of the concentrate being the extraction solvent and/or an additional solvent.

The inventors have indeed shown that the rose extracts according to the invention included micronutrients and natural compounds of interest for the skin, and that the combined use of a polar (aqueous) extract of rose and an apolar (oil) extract of rose made it possible, in the same cosmetic composition, to provide all the nutritional and sensory benefits of the rose.

As natural polar compounds of interest present in the aqueous extract of rose according to the invention, particular mention may be made of:
vitamins B3 and B5, antioxidants,
minerals and trace elements, combined with skin protection and repair systems,
sugars (sucrose, glucose, fructose) central to the skin's energy processes,
flavonoid-type polyphenols (such as kampferol and quercetin derivatives), with anti-free radical activities, and
amino acids, source of vitality and protein components for the cell.

As natural non-polar compounds of interest present in the oil extract of rose according to the invention, particular mention may be made of:
fat-soluble vitamins such as vitamin E or α-tocopherol, antioxidant,
omega-3, 6 and 9 and fat acids involved in resilience and epidermal tissue dynamics,
antioxidant phytonutrients, preserving and strengthening the skin,
sterols β-sitosterol and stigmasterol, modulators of immunity and of cholesterol metabolism, and
apolar phenols with antioxidant and antimicrobial properties.

The aqueous and oil extracts of rose according to the invention can be obtained by various processes known to the skilled person, in particular those described below.

According to a particular and preferred embodiment, the extraction processes adapted to the invention described below will use as plant material flowers of the Evanrat variety, preferably the Jardin de Granville® rose, freshly harvested, i.e. within 24 hours and frozen spread in thin layers at approximately −25° C.

### Aqueous Extract of Rose (Also Called ‘Cryoextract’ in the Illustrative Examples of the Invention)

In the description, the terms aqueous extract and polar extract and hydrophilic extract of rose are used interchangeably. The aqueous extract of rose is advantageously obtained using a cosmetically acceptable polar solvent.

According to the invention, ‘aqueous extract of rose’ means that the polar (hydrophilic) compounds of the rose flowers have solubilized and/or have been extracted in a polar solvent.

Prior to the extraction step itself, the flowers may have been dried and/or ground. Advantageously, freshly harvested flowers are used.

The extract can be prepared by various extraction processes known to the skilled person, involving steps of grinding the plant material, dispersing the ground material in a polar solvent, separating the soluble and insoluble phases by filtration, concentration and possible re-solution.

According to the present invention, the term “polar solvent” means that the solvent has a polarity index of 4 or higher. The polarity index is a quantity calculated on the basis of thermodynamic quantities (solubility and state change) that shows the more or less polar nature of a molecule. For solvent polarity indices, see the article by L. R. Snyder: Classification of the solvent properties of common liquids; Journal of Chromatography, 92 (1974), 223-230.

The preferred polar solvents are those consisting of a compound containing at least one polar covalent bond of type O—H. As a particularly preferred polar solvent, a solvent or solvent mixture chosen from water, C₁-C₄ alcohols, such as ethanol, glycols, such as ethylene glycol, glycerol, butylene glycol and propylene glycol, and mixtures thereof are chosen. Advantageously, water, ethanol or a mixture thereof will be used.

According to a particular embodiment, the plant material is extracted using a hydro-alcoholic mixture, preferably a mixture of water and ethanol. In particular, the plant material is extracted using a solvent consisting of water and ethanol, with ethanol representing from 50% v/v to 99% v/v of the water/ethanol mixture. Preferably the plant material is extracted using a mixture of 30% v/v water and 70% v/v ethanol. Extraction can be carried out hot by reflux or by maceration at room temperature.

Ultrasound during extraction is used advantageously to improve the mass efficiency of the extraction.

The extraction process advantageously includes a filtration step to separate the liquid phase from the spent plant material.

The extraction and filtration cycle can be reproduced several times in order to exhaust the plant material of substances with an affinity for the extraction solvent

The extraction process may also include at least one bleaching and/or purification step, for example in the form of treatment of the extract with a solution of at least one polar solvent in the presence of activated carbon particles. In particular, chlorophyll extracted by the solvent is eliminated.

The extraction process can also be completed by a step of partial or total removal of the extraction solvents.

It is advantageous to concentrate the extract by removing some of the solvent or mixture from the extraction solvent.

This can result in an aqueous concentrate without a significant amount of organic solvents or, by removing the entire extraction solvent, a dry residue.

Alternatively, the product of the extraction step can be freeze-dried or atomized to form a powder.

According to a particularly preferred embodiment, the aqueous extract of rose flowers according to the invention is obtained according to a process making it possible to increase the extraction yield and to enrich the extract with water-soluble products usually contained in plant juices, in particular sugars, mineral salts, proteins, beneficial for the micronutrition of the skin.

Thus, according to a particular and preferred embodiment of the invention, the aqueous extract of rose is obtained by a process of cryo-grinding and cold extraction of the rose flowers, allowing a preservation of heat-sensitive molecules.

Such a process is described in particular in application EP0425391 incorporated by reference.

This process comprises in particular the following steps:
first grinding of rose flowers at a temperature between −10° C. and −40° C.,
second grinding of the rose fractions obtained in the preceding step, at a temperature between −40° C. and −100° C., in the presence of liquid nitrogen
sieving of the fractions obtained in the preceding step, using a sieve with a grain size ranging from 2 mm to 100 μm and preferentially less than 500 μm, in particular from 100μ to about 500μ,
pressing of the fractions recovered in the preceding step, and reduced to a temperature of 0° C.±5° C., then subjected to the following cycle of operations: freezing to a temperature between −10° C. and −40° C., suspension of these frozen fractions in a quantity of water approximately equal to the quantity of liquid obtained in the preceding step, pressing of the fractions in suspension in the water and reduced to a temperature of 0° C.±5° C.,
filtration of the quantities of liquids obtained and recovery of the filtrates
concentration of the filtrates by cold water removal
advantageously freezing the concentrated solutions obtained in the preceding step.

According to a particular and preferred embodiment, the aqueous extract of rose according to the invention, also called “cryoextract” in the illustrative examples below, comprises from 0.5% to 10% by weight of dry matter (active matter) of rose extract, and 90% to 99.5% by weight of a 50:50 mixture of water and glycerol. Preferably, the ‘cryoextract’ comprises 0.5% to 1.5% by weight of dry matter (active substance), and 98.5% to 99.5% by weight of a 50:50 mixture of water and glycerol.

The INCI name of this aqueous extract of rose is Water, Glycerine, Rose Extract.

It can also be found under the name Rosa Hybrid Flower Extract, Water, Glycerine.

According to a particular and preferred embodiment of the invention, the cosmetic composition according to the invention is characterized in that the aqueous extract of rose comprises an extract of rose flowers in a polar solvent, in particular in a weight ratio of 0.5:99.5 to 10:90 (plant extract: polar solvent) and is present in the composition in a content ranging from 0.1% to 10%, in particular from 0.5% to 5%, and according to a particular embodiment from 1% to 4% by weight of raw material based on the total weight of said composition.

The aqueous extract of rose according to the invention, in particular obtained by the cryo-grinding process described above, advantageously includes polar micronutrients, such as vitamins (vitamin B3, vitamin B5), trace elements (zinc, copper, iron, manganese), sugars (sucrose, glucose, fructose), an amino acid (aspartic acid) and flavonoid-type polyphenols (such as kampferol and quercetin derivatives).

### Oil Extract of Rose (Also Called ‘Satin Oil’ in the Illustrative Examples)

In the description, the terms oil extract and apolar extract and lipophilic extract of rose are used interchangeably.

The oil extract of rose according to the invention is inspired by the theory of the polar paradox of antioxidants of Porter and Frankel or physicochemical behaviour of antioxidants in continuous oil system and dispersed system allowing hydrophilic compounds (antioxidants or phenols for example) to become miscible in oil compounds containing for example phospholipids (molecules with polar functions) under physicochemical actions produced by ultrasound and microwaves.

More simply, it is a matter of using an oil or a fat, and in particular a vegetable oil, as a biologically active agro-solvent, to selectively extract molecules from a plant, in particular a rose, with a powerful technology based on physical principles, to obtain a concentrated oil complex, a stable vector ready to be formulated.

According to the invention, ‘oil extract of rose’ means in particular that the apolar (lipophilic) compounds of rose flowers have solubilized and/or have been extracted in an oil.

The oil extract of rose according to the invention can be obtained according to the classical methods known to the skilled person, such as methods of maceration in an oil solvent, such as those described in WO2008/132127 or FR2693906, optionally under microwave radiation as described in FR2694300 to reduce the extraction time.

According to a particular and preferred embodiment of the invention, the oil extract of rose is obtained by a dynamic extraction method (also called “dynamic enfleurage”) which is done by energetic activation of the plant and the carrier oil to have an active mass transfer, with a pumping effect from the most concentrated medium to the least concentrated. The process combines multi-steps (cryo-grinding, ultrasound, microwave, filtration) of short duration (each step <15 min) and inerting in nitrogen and protected from light during transformation limit oxidation phenomena.

This opens the plant cells, disorganizes the medium to make extractable the insoluble molecules usually found in oils, they become miscible with the oil support according to a dynamic rearrangement.

The inventors indeed observed that the total phenol content is advantageously higher with the “dynamic enfleurage” extraction method compared to a “classic enfleurage” extraction method (classic maceration), i.e. 25 to 40 times higher. This method makes it possible to entrain the phenols contained in the rose compared to a traditional enfleurage method and also with a polar solvent such as water.

Such a process is described in particular in application WO2010/112760 incorporated by reference. In particular, the process comprises:
at least one step a) of mixing and impregnating the solid raw material with a natural fat at a temperature above the melting point of the oil and under an atmosphere free or essentially free of oxygen,
at least one step b) of heating the mixture to a high temperature for a very short period of time and under an atmosphere free or essentially free of oxygen, one using microwaves,
at least one step c) of microdispersing the material to be extracted and optionally rupturing the cells of the raw material in the natural fat at a temperature above the melting point of the fat and under an atmosphere free or essentially free of oxygen, one using ultrasound,
step c) that can be performed before, during or after step b).

The raw material is preferably ground beforehand at low temperature, between −20° C. and −80° C. (cryo-grinding).

Advantageously, in step c) or just before, an oxygen trapping or reducing compound, a compound to regenerate in reduced form the tocopherols of the oil and the phenolic compounds extracted in the oil by the process or a pro-oxidant metal chelator is added, these compounds helping to improve the oxidative stability of the final product.

As fats or vegetable oils that can be used as extraction carriers for the oil extract according to the invention, particular mention may be made of the following vegetable oils and fats:
vegetable oils such as: deodorized sunflower oil, virgin sweet almond oil, virgin rosehip oil, avocado oil, safflower oil, camelina oil, jojoba oil, borage oil, grape seed oil, argan oil, nigella oil, pumpkin seed oil, or perilla oil, and mixtures thereof,
butters such as: murumuru butter, mango butter, shea butter, and mixtures thereof,
vegetable waxes such as: carnauba wax, beeswax, candelilla wax, jojoba wax, and mixtures thereof,
and mixtures thereof.

Preferentially, organic deodorized oleic sunflower oil, which is orange-yellow in colour, will be used.

According to a preferred embodiment of the invention, the weight ratio between the raw material (rose flowers) and the oil in the starting mixture used in step a) is between 1:0.5 and 1:10, preferably between 1:1 and 1:5 expressed as mass:mass of oil or mass:volume of oil.

According to a preferred embodiment, the oil extract of rose according to the invention is obtained according to the following process: the fresh frozen flowers are cryo-ground and mixed with the organic deodorized oleic sunflower oil, in particular in a dry plant to oil ratio ranging from 1:10 to 1:5, then different successive extraction steps are made by extraction using microwaves and then by ultrasound. A centrifugal filtration step results in a precious oil whose stability has been preserved because each step has been carried out meticulously under nitrogen inerting. We obtain the oil concentrate of rose or oil extract of rose which is a “dynamic” enfleurage of the Jardin de Granville® rose.

This raw material can be used in this manner or combined with other materials to form new oil complexes.

According to a preferred embodiment, the oil extract of rose according to the invention comprises 98.5%-99.5% organic deodorized oleic sunflower oil and 0.5-1.5% rose extract (dry matter or active matter from the rose extract).

The INCI name of this oil extract of rose is Rose extract and Helianthus annuus (sunflower) seed oil.

The INCI name Rosa Hybrid Flower Extract, Helianthus annuus (sunflower) seed oil can also be found.

According to a particular and preferred embodiment of the invention, the cosmetic composition according to the invention is characterized in that the oil extract of rose comprises an extract of rose flowers in a vegetable oil, preferably a sunflower oil, in particular in a weight ratio of 1:99 to 10:90 (plant extract to oil) and is present in the composition in a content ranging from 0.1% to 10%, in particular from 0.5% to 5%, and according to a particular embodiment from 1% to 2% by weight of raw material based on the total weight of said composition.

Analysis of the composition of this extract shows that it is rich in non-polar micronutrients and in particular:
fatty acids (majority compounds)
   C16:0 (palmitic acid)
   C18:0 (stearic acid)
   C18:1 (oleic acid—omega 9)
   C18:2 (linoleic acid—omega 6)
   C20:0 (arachidic acid)
   C20:1 (eicosenoic acid—omega 9)
   C22:0 (behenic acid)
fat-soluble vitamins (α-tocopherol)
sterols β-sitosterol and stigmasterol, and
apolar phenols.

According to a particular and preferred embodiment of the invention, use is made of at least:
one aqueous extract of rose flowers of the Evanrat or Jardin de Granville® rose variety obtained according to the cryoextraction process described in application EP0425391; in particular an extract comprising 0.5% dry matter in 99.5% of a water/glycerol mixture (called ‘cryoextract’ in the illustrative examples) and
one oil extract of rose flowers of the Evanrat or Jardin de Granville® rose variety obtained by the dynamic enfleurage process as described in application described in application WO2010/112760; in particular an extract comprising from 0.5% to 1.5% dry matter in 98.5-99.5% organic deodorized oleic sunflower oil (called ‘satin oil’ in the following illustrative examples).

The content of aqueous extract of rose in the final cosmetic composition will generally range from 0.1% to 10%, in particular from 0.5% to 5%, and according to a particular embodiment from 1% to 4% by weight of the raw material described above, based on the total weight of said composition. For a raw material comprising 0.5% by weight of dry extract of rose, this is equivalent to 0.0005% to 0.05% by weight, in particular from 0.0025% to 0.025%, and according to a particular embodiment from 0.005% to 0.02% by weight of dry (active) matter based on the total weight of the composition.

The content of oil extract of rose in the final cosmetic composition will generally range from 0.1% to 10%, in particular from 0.5% to 5%, and according to a particular embodiment from 1% to 2% by weight of the raw material described above, based on the total weight of said composition. Since the oil extract represents 100% dry extract, for a raw material containing 1% rose extract, this represents an equivalent of 0.001% to 0.01% by weight of active matter from rose extract, in particular from 0.005% to to 0.05% by weight and according to a particular embodiment from 0.01% to 0.02% by weight of active matter from rose extract.

Advantageously the weight ratio between the aqueous extract and the oil extract of rose flowers in the final cosmetic composition will range from 5:1 to 1:1, preferably 3:1 in active matter.

### Galenic

The cosmetic composition of the invention may be in any galenic form suitable for topical application to the skin, such as serum-oil, oil-in-water emulsion, water-in-oil emulsion, multiple emulsion, or aqueous gel.

Advantageously, use will be made of a cosmetic composition comprising at least one aqueous phase and at least one fat or oil phase in which each rose extract according to the invention, depending on its polarity, can be optimally dispersed and thus fully express its effects when the composition is applied to the skin.

Thus, according to a particular and preferred embodiment of the invention, the cosmetic composition of the invention comprises at least one aqueous phase comprising said aqueous extract of rose and at least one fat or oil phase comprising said oil extract of rose.

The preferred embodiments according to the invention are the methods of obtaining a dispersion of a fat phase containing the oil extract of rose in an aqueous phase containing the aqueous extract of rose. According to a particular embodiment, it will be an oil-in-water emulsion in which the size of the oil droplets or beads may vary from a few nanometres to a few millimetres depending on the desired effect.

The skilled person will be able to choose the appropriate method by referring in particular to the following publication for the definition and size of the different types of emulsion: Emulsion Formation, Stability, and Rheology, Prof. Dr. Tharwat F. Tadros, Published Online: 29 Jan. 2013, DOI: 10.1002/9783527647941.ch1, http://onlinelibrary.wiley.com/doi/10.1002/9783527647941.ch1/summary.

The skilled person will be able to choose the method of measuring particle size and distribution according to the expected size range, either by dynamic or static light scattering for droplets ranging from 5 nm to 100 μm, with particular reference to the following publication: “Light Scattering by Small Particles” by H. C. van de Hulst, 24 May 1982, or by image analysis for droplets or spheroids above 100 μm.

According to a particular embodiment, the cosmetic composition according to the invention is in the form of a dispersion of a fat phase in an aqueous phase, in particular chosen from the group consisting of emulsions, macroemulsions, nanoemulsions, microemulsions, Pickering emulsions, solid fat dispersions, or oil droplet dispersions stabilized by a polymeric membrane.

Exemplary embodiments are described below, without this being restrictive (the average diameter of the oil droplets in the aqueous phase is indicated in brackets).

According to a first embodiment, the cosmetic composition of the invention can have the form of an oil-in-water emulsion or macroemulsion (0.1-100 μm).

According to a second embodiment, the cosmetic composition of the invention can have the form of an oil-in-water nanoemulsion (20-100 nm).

According to a third embodiment, the cosmetic composition of the invention can have the form of a microemulsion or micellar emulsion (5-50 nm).

These three systems are generally kinetically or thermodynamically stabilized by surfactants preferably of high HLB (>8 at 25° C.) or associative polymers such as associative polyurethane (Adekanol GT700).

According to a fourth embodiment, the cosmetic composition of the invention can have the form of a Pickering emulsion stabilized by silica or clay particles such as kaolinite or montmorillonite.

According to a fifth embodiment, the cosmetic composition of the invention can have the form of a solid dispersion of fat, spherical or spheroidal (50 μm to 10 mm), as described in particular in patent application FR2649608. According to this embodiment, the fat phase is heated to complete melting and mixed in an aqueous phase heated to the same temperature and then cooled rapidly under stirring.

According to a particular and preferred sixth embodiment of the invention, the cosmetic composition of the invention is in the form of a dispersion of droplets of a first oil phase in a second aqueous phase, the droplets or ‘beads’ being stabilized by a polymeric membrane on the surface (interfacial coacervation between cationic and anionic polymers) (500 μm to 5 mm), as described in patent application FR2972371, incorporated by reference.

Each droplet comprises a core formed of an oil phase and a shell formed of a coacervate layer interposed between the oil phase and the aqueous phase, said layer being created by interaction between a first precursor lipophilic polymer contained in the oil phase and a second precursor hydrophilic polymer contained in the aqueous phase at the interface of the two phases.

According to a particular embodiment, the oil phase comprising the oil extract of rose further comprises a lipophilic polymer ionizable in contact with an aqueous phase, for example a polymer containing a silicone and containing an ionizable functional group, the lipophilic polymer being advantageously a dimethicone derivative, such as amodimethicone and derivatives thereof.

According to another particular embodiment, the lipophilic polymer is supplied by an oil phase, called the reaction phase, other than the oil phase containing the oil extract of rose.

And the aqueous phase includes an acrylic hydrophilic polymer such as a copolymer of acrylic acid or maleic acid and at least one other monomer, such as acrylamide, alkyl acrylates, C₅-C₈ alkyl acrylates, C₁₀-C₃₀ alkyl acrylates, C₁₂-C₂₂ alkyl methacrylates, methoxypolyethyleneglycol methacrylates, hydroxyester acrylates.

According to a particular and preferred embodiment, the step of stiffening the droplets is based on the formation of a coacervate at the interface between the polyacrylic acid contained in the aqueous phase and an amino-silicone (amodimethicone) provided by the oil phase, after droplet formation. The meeting of these two polymers causes the coacervation and stiffening of the membrane around the droplets.

This formulation is advantageous in that the oil extract of rose is microencapsulated in oil droplets protected by an ultrathin membrane, stabilized without surfactant. Upon application, the membrane bursts and disappears. There is therefore no interface between water and oil, which becomes immediately available on the skin without hindrance. The alternation of the two phases creates a very particular sensory transformation with a unique touch and allows the rose extracts, protected in their respective phases, to be directly bioassimilable by the skin.

Thus, according to a particular and preferred embodiment, the cosmetic composition of the invention is in the form of an oil-in-water emulsion, a silicone-in-water emulsion, a multiple emulsion, or preferably a dispersion of droplets (or “beads”) of oils stabilized by a polymeric membrane suspended in a preferably gelled aqueous phase.

The aqueous phase generally represents from 1% to 99% by weight, based on the total weight of said composition.

The composition is preferably intended to be applied to the face and takes the form of a skin care cream, a facial fluid, a facial skin care gel, for example.

### Aqueous Phase

The aqueous phase of the composition according to the invention includes water and optionally a water-soluble solvent.

According to the invention, ‘water-soluble solvent’ means a compound that is liquid at room temperature and miscible with water (miscibility in water greater than 50% by weight at 25° C. and atmospheric pressure). Particular mention may be made of:
lower C₁-C₅ monoalcohols such as ethanol, isopropanol and mixtures thereof;
C₂-C₈ glycols such as ethylene glycol, propylene glycol, 1,3-butylene glycol, dipropylene glycol, and mixtures thereof;
C₂-C₃₂ polyols such as polyglycerols, polyethylene glycols, and mixtures thereof,
   and mixtures thereof.

It may also include hydrophilic gelling agents, antioxidants, preservatives and mixtures thereof.

As hydrophilic gelling agents, particular mention may be made of polyacrylic acids such as those with the INCI name ‘carbomer’ or the trade name Carbopol®, carboxyvinyl polymers, associative thickeners of acrylic or polyurethane type, polysaccharide gelling agents such as alginates, xanthan gums, carrageenan gums, agar gums, cellulose derivatives, gelatin, mineral gelling agents such as bentones or modified silicas, and mixtures thereof.

According to a particular embodiment, the cosmetic composition of the invention includes a gelled aqueous phase, in particular gelled by the presence of at least one polyacrylic acid polymer.

### Fat or Oil Phase

The cosmetic composition of the invention includes a fat (solid fat) or oil phase.

“Oil phase” means an oil or a mixture of intermiscible oils. “Oil” means, in the sense of the invention, a fat, not soluble in water, liquid at 25° C. and 0.1 MPa, and preferably non-volatile having a vapour pressure, at 25° C. and 0.1 MPa, not zero less than 2.6 Pa, preferably less than 0.13 Pa.

An oil phase according to the invention may include hydrocarbon oils, silicone oils, fluorinated oils or not, and mixtures thereof.

These oils can be volatile or non-volatile, vegetable, mineral or synthetic.

According to the invention, ‘hydrocarbon oil’ means an oil containing mainly hydrogen and carbon atoms.

According to the invention, ‘silicone oil’ means an oil containing at least one silicon atom, and in particular at least one Si—O group.

According to the invention, ‘fluorinated oil’ means an oil containing at least one fluorine atom.

As non-volatile hydrocarbon oils, particular mention may be made of hydrocarbon oils, vegetable hydrocarbon oils, C₁₀-C₄₀ synthetic ethers, C₁₀-C₄₀ synthetic esters, C₁₂-C₂₆ fatty alcohols, higher C₁₂-C₂₂ fatty acids, and mixtures thereof.

As non-volatile silicone oils, particular mention may be made of phenylated silicone oils, non-phenylated silicone oils, and mixtures thereof.

The oils may be present in the composition of the invention in a content ranging from 1% to 95% by weight based on the total weight of the composition.

The fat or oil phase may also include lipophilic gelling agents, film-forming polymers, surfactants, antioxidants and mixtures thereof.

According to a particular embodiment, the aqueous phase comprises at least one polyacrylic acid and the oil phase comprises at least one amino-silicone (amodimethicone). The combination of these two polymers causes the coacervation and stiffening of the membrane around the droplets, allowing them to be suspended in the aqueous phase, which can itself be advantageously transparent and gelled.

According to a particular embodiment, the cosmetic composition of the invention comprises less than 5% by weight surfactant, in particular less than 2% by weight surfactant, preferably less than 1% by weight surfactant and more preferably is free of surfactant.

The composition of the invention may also include any additive commonly used in cosmetics such as UV filters, antioxidants, fragrances, cosmetic active agents, such as emollients, moisturizers, vitamins, anti-ageing agents, lightening agents, and mixtures thereof.

According to a particular and preferred embodiment of the invention, the cosmetic composition according to the invention is in the form of a dispersion of oil droplets comprising the oil extract of rose of the Evanrat or Jardin de Granville® rose variety in a gelled aqueous phase comprising the aqueous extract of rose of the Evanrat or Jardin de Granville® rose variety.

The oil phase of such a composition comprises at least one amino-silicone (amodimethicone) and the aqueous phase at least one polyacrylic acid.

And preferably said cosmetic composition comprises at least:
one aqueous extract of rose flowers of the Evanrat or Jardin de Granville® rose variety obtained according to the cryoextraction process described in application EP0425391; in particular an extract comprising 0.5% dry matter in 99.5% of a water/glycerol mixture (called ‘cryoextract’ in the illustrative examples) and
one oil extract of rose flowers of the Evanrat or Jardin de Granville® rose variety obtained by the dynamic enfleurage process described in application described in application WO2010/112760; in particular an extract comprising from 0.5% to 1.5% dry matter in 98.5-99.5% organic deodorized oleic sunflower oil (called ‘satin oil’ in the following illustrative examples).

According to a particular embodiment, the cosmetic composition of the invention does not include any other rose extract than the rose extracts described according to the invention.

### Cosmetic Process

The invention also relates to a cosmetic process to promote the natural rhythmic process of skin cells and/or the micronutritional balance of the skin, comprising the application to the skin, in particular to the face and/or neck, of a cosmetic composition as previously defined according to the invention.

The composition can be applied to the body, face and/or neck. According to a particular embodiment, the composition is applied to the face and/or neck.

Advantageously, and by virtue of the knowledge acquired by the Applicant on the role of clock genes and their circadian oscillations in skin cells, the cosmetic composition of the invention can be applied to the skin of the face and/or neck in the morning, to optimize skin care and to obtain an improved benefit on the skin barrier and skin resistance.

### Cosmetic Uses

The invention also relates to the non-therapeutic cosmetic use of at least an effective amount of at least one aqueous extract of rose and at least one oil extract of rose in a cosmetic composition, as an agent to promote and/or improve the natural rhythmic process of skin cells, the micronutritional balance of the skin, the lipid metabolism of the skin and/or the skin barrier function.

According to a particular embodiment, the effective amount of at least one aqueous extract of rose and at least one oil extract of rose stimulates the expression of epidermal clock genes, and the expression of epidermal clock target genes, involved in particular in lipid metabolism, the skin barrier, cell differentiation, cell communication and/or cell cohesion.

In particular, the aqueous extract of rose according to the invention stimulates the expression of the clock genes Cryptochrome Circadian Clock 2 CRY2, Period 1 PER1 and Period 3 PER3 and the oil extract of rose according to the invention stimulates the expression of the clock gene Period 2 PER2.

The aqueous extract of rose according to the invention stimulates the expression of the genes keratin 10 KRT10 and desmoglein 1 DSG1 and the oil extract of rose according to the invention stimulates the expression of the genes ceramide-synthase 3 CERS3, calmodulin 3 CALM3, keratin 1 KRT1, gap-junction alpha-1 protein GJA1/connexin 43 Cx43 and desmocollin 3 DSC3.

The aqueous and oil extracts of rose used according to the invention are as described above. Preferably, the following will be used:
an aqueous extract of rose flowers of the Evanrat or Jardin de Granville® rose variety obtained according to the cryoextraction process described in application EP0425391; in particular an extract comprising 0.5% dry matter in 99.5% of a water/glycerol mixture (called ‘cryoextract’ in the illustrative examples) and
an oil extract of rose flowers of the Evanrat or Jardin de Granville® rose variety obtained by the dynamic enfleurage process described in application described in application WO2010/112760; in particular an extract comprising from 0.5% to 1.5% dry matter in 98.5-99.5% organic deodorized oleic sunflower oil (called ‘satin oil’ in the following illustrative examples).

The invention will now be illustrated in the following figures and non-limiting examples.

### FIGURES

FIG. 1: Activation of CRY2 gene transcription in NHK treated with rose extracts.

FIG. 2: Activation of PER 1 gene transcription in NHK treated with rose extracts.

FIG. 3: Activation of PER 3 gene transcription in NHK treated with rose extracts.

### EXAMPLES

### Materials and Methods

The plant material used to obtain the aqueous and oil extracts of rose illustrated in these examples is rose flowers (petals) of the Evanrat variety, in particular Jardin de Granville® rosebush flowers, available from nurseries.

### Aqueous Extract of Rose (‘Cryoextract’)

The plant material used is rose flowers (petals) of the Evanrat variety, in particular Jardin de Granville® rosebush flowers available from nurseries.

The aqueous extract of rose flower is obtained by the cryoextraction process described above, in particular by the process described in patent application EP0425391. A cryoextract is obtained comprising 0.5% by weight dry matter (active matter), 49-50% by weight water, 49% by weight glycerol and preservatives qs 100%. The INCI name of this aqueous extract of rose is Water, Glycerine, Rose Extract or Rosa Hybrid Flower Extract, Water, Glycerine.

### Oil Extract of Rose (‘Satin Oil’)

The plant material used is rose flowers (petals) of the Evanrat variety, in particular Jardin de Granville® rosebush flowers available from nurseries.

The oil extract of rose flower is obtained by the dynamic enfleurage process described above, in particular by the process described in patent application WO2010/112760. The result is an oil extract ‘satin oil’ comprising 0.5-1.5% by weight rose dry matter (active matter), and 98.5-99.5% by weight organic deodorized oleic sunflower oil.

The INCI name of this oil extract of rose is Rose extract and Helianthus annuus (sunflower) seed oil or Rosa Hybrid Flower Extract, Helianthus annuus (sunflower) seed oil.

### Example 1: Effect of the Aqueous Extract of Rose Flower on the Expression of Epidermal Clock Genes (PER1, PER 3, CRY2)

A study was carried out on the impact of the aqueous extract of rose on the expression of genes involved in the molecular clock in normal human keratinocytes, in particular the genes Period 1 (PER1), Period 3 (PER3), Cryptochrome Circadian Clock 2 (CRY2), involved in metabolic pathways for skin resistance, skin nutrition such as lipid metabolism, glucose metabolism, calcium homeostasis (involved in epidermal differentiation).

Normal human keratinocytes (NHK) are cultured and then treated with the 2 rose extracts. After NHK treatment, a TaqMan low-density array (TLDA) study on the genes studied is carried out using cDNA obtained after reverse transcription of the total extracted RNA.

The oil extract of rose is tested at 25 μg/mL and 10 μg/mL (DMSO excipient for compatibility of the oil extract with the cell culture medium).

The aqueous extract of rose is tested at 1% and 3% (water excipient).

### NHK Culture

Normal human keratinocytes are derived from a skin sample from plastic surgery. The cells are cultured in the complete EpiLife medium at P5 with a seeding density of 50,000 cells per well, in 12-well plates. At subconfluence, the cells are treated 24 hours with the doses of rose extracts described above.

### Real-Time Quantitative RT-PCR

Obtaining Total RNA

The cell culture medium is removed, and 250 μL of RLT lysis buffer (provided in the NucleoSpin RNA Trace Kit, Macherey-Nagel) is added. The cells are scraped with a cell scraper and then the cell lysate is recovered in a 1.2 mL deep-well (provided in the NucleoSpin RNA Kit). Total RNA is extracted according to the defined protocols.

The total RNA solutions obtained are measured and their quality verified using a microplate reader, the SPECTROstar Nano (BMG Labtech) coupled to the Microlab STAR. This device is connected to the computer controlling the automated system and has the specific software for the analysis of results (MARS software). The technique requires a 384-well microplate (LoBase), a positive control (RNA 250, AM7155, Thermo Fisher) to validate the pipetting performed by the robotic system as well as the values generated by the SPECTROstar Nano reader.

Synthesis of Complementary DNA

The reverse transcription (RT) kit used is the High Capacity Reverse Transcription Kit (Thermo Fisher). It was used according to the protocol provided. Total RNA (500 ng) is diluted in water to a final volume of 25 μL. It is then incubated for 10 minutes at 25° C. and then 2 hours at 37° C. in the presence of 25 μL of High Capacity Reverse Transcription Kit 2× reaction mixture previously prepared as indicated below. The different incubations are done within the TRobot (Biométra).

TABLE 1
High Capacity Reverse Transcription Kit
2X reaction mixture for 1 reaction

| Reagents | RT buffer | dNTP | Primer | RNase OUT | RT | H2O |
|---|---|---|---|---|---|---|
| Volume | 5 μL | 2 μL | 5 μL | 0.5 μL | 2.5 μL | 10 μL |

PCR-TaqMan Low-Density Array

Each RT (15 μL) is mixed with 60 μL of water and then 75 μL of TaqMan Gene Expression Master Mx (Thermo Fisher item 4369510), containing the DNA polymerase, is added. After homogenization, 100 μL is deposited on microfluidic cards containing the probes corresponding to the clock genes tested (Table 2 below), the latter are centrifuged and then sealed. The CD-ROM corresponding to the profile of the genes deposited on the plates is loaded into the SDS 2.3 software, specifying the location of each gene on the card. The control gene (or “endogenous” gene) to be used for normalizing the results should be indicated before the start of PCR. This is done according to the protocol provided by Applied Biosystems in the ABI Prism 7900HT Sequence Detection System. The qPCR steps are 2 min at 50° C., 10 min at 94.5° C. then 30 s at 97° C. and 1 min at 59.7° C. for 40 cycles.

TABLE 2
List of genes on the microfluidic card.

| | | RefSeq GenBank |
|---|---|---|
| Gene | TaqMan item no. | accession number |
| Per1 | Hs01092603_m1 | NM_002616.2 |
| Per3 | Hs00997925_m1 | NM_016831.2 |
| Cry2 | Hs00323654_m1 | NM_021117.3 |
| Beta-2-M | Hs00187842_m1 | NM_004048.2 |
| (control gene) | | |

### Statistical Analyses

Real-time quantitative PCR can be used if its effectiveness is between 90% and 110%. For each sample, the number of cycles at which the signal appears is determined by the SDS 2.3 software. For the same test, the expression levels of the transcripts of interest obtained are normalized to the value obtained for the housekeeping gene beta-2-microglobulin. This gene, whose expression is constitutive and invariant, makes it possible to avoid any variations induced during the experiment (total RNA assay, pipetting, reverse transcription step, PCR in the apparatus).

In the RT-PCR TLDA method, quantification is performed using the ΔΔCt comparative method. The relative quantification (RQ) values obtained correspond to the amplitude level (x times more or less than the control) of the expression compared to our control, here the non-irradiated one. The RQ is obtained by the following calculation where the control is equal to 1:

RQ=2^{-ΔΔCt}=2^{-(ΔCt treated-ΔCt untreated)}
ΔCt treated=Ct target gene treated−Ct housekeeping gene treated
ΔCt untreated Ct target gene untreated−Ct housekeeping gene untreated

In order to evaluate statistically significant changes in transcriptional activity, we will use Student's t-test. Each condition is carried out in triplicate (3 untreated and 3 treated under the same conditions). Fischer's F-test is first applied by comparing the two data matrices. When the value is greater than α=0.05 then the variance for Student's t-test is 2, when the Fischer F-test is less than α=0.05 then the variance will be 3. The transcriptional variations selected will be those with a Student's t-test lower than α=0.05.

The results are presented as an average with n=3. Student's t-test was used to compare the effect between treated and untreated cells.

The results are considered significant for p<0.05(*) or p<0.01(**).

### Effect on CRY2

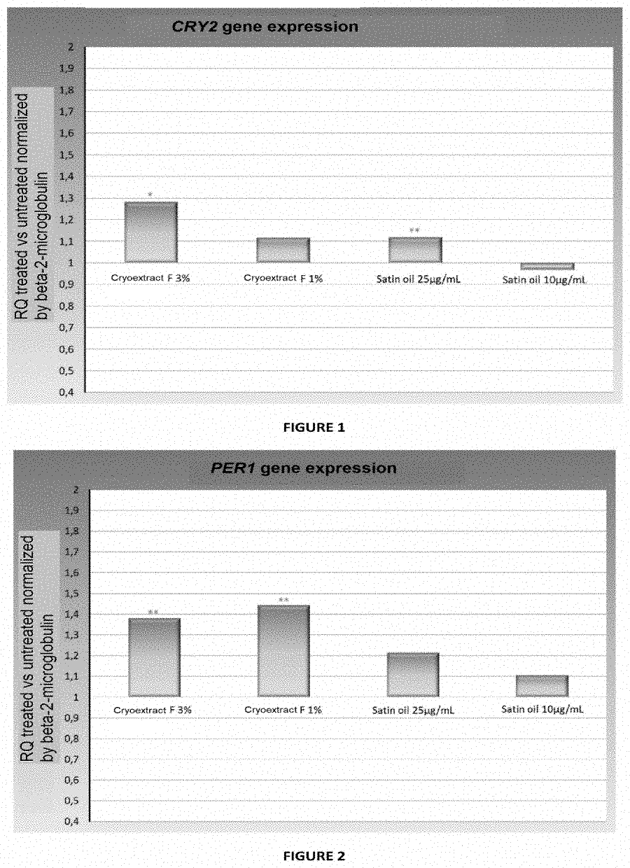

FIG. 1 shows a significant increase in transcriptional activity for the aqueous extract of rose flower at 3% which increases expression of the CRY 2 gene by 28%, and the oil extract of rose flower at 25 μg/mL which increases expression of the CRY 2 gene by 12%.

### Effect on PER 1

FIG. 2 shows a significant increase in the expression of the PER 1 gene.

### Effect on PER 3

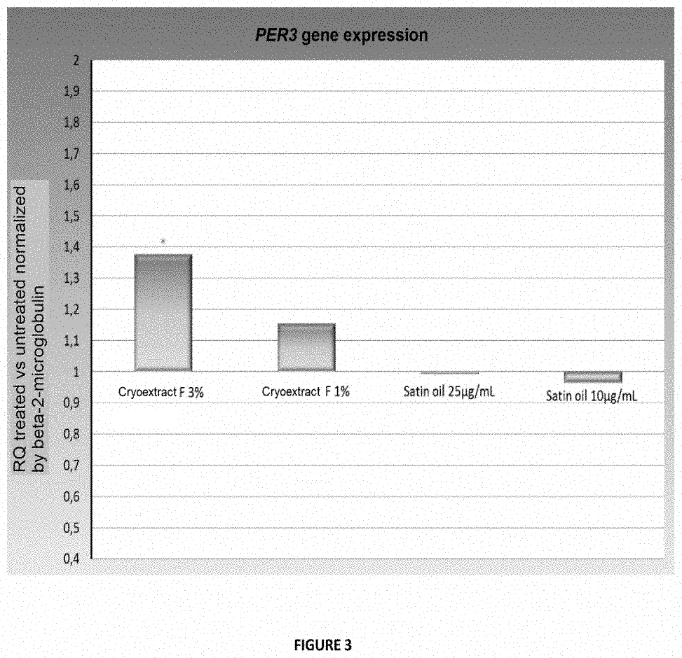

FIG. 3 shows a significant increase in transcriptional activity for the aqueous extract of rose flower at 3% which increases gene expression by 38%.

The aqueous extract of rose flower alone therefore increases the effect of the CRY2, PER1, and PER3 genes.

### Example 2: Effect of the Oil Extract of Rose Flower on the Expression of the Clock Gene PER 2

An oil extract of rose flower at 1 μg/mL (DMSO excipient) was tested on NHK according to a protocol similar to that described in example 1.

TABLE 3
List of genes on the microfluidic card.

| | | RefSeq GenBank |
|---|---|---|
| Gene | TaqMan item no. | accession number |
| Per2 | Hs00256143_m1 | NM_022817.2 |
| Beta-2-M | Hs00187842_m1 | NM_004048.2 |
| (control gene) | | |

A 21% increase in the expression of the PER 2 gene is observed.

The oil extract of rose flower therefore has an effect complementary to the aqueous extract of rose on the expression of clock genes.

### Example 3: Effect of Oil Extract of Rose Flower on Gene Expression in Normal Human Keratinocytes

An oil extract of rose flower at 1 μg/mL (DMSO excipient) was tested on NHK according to a protocol similar to that described in example 1.

TABLE 4
List of genes present on the microfluidic card.

| | | | RefSeq GenBank |
|---|---|---|---|
| | Gene | TaqMan item no. | accession number |
| Differentiation/ | CERS3/LASS3 | Hs00698859_m1 | NM_178842.4 |
| Skin barrier/ | CALM3 | Hs00270914_m1 | NM_005184.2 |
| Lipids | KRT1 | Hs00196158_m1 | NM_006121.3 |
| Accession/ | GJA1/CX43 | Hs00748445_s1 | NM_000165.4 |
| Cohesion/ | DSC3 | Hs00170032_m1 | NM_001941.4 |
| Communication | | | |
| | Beta-2-M | Hs00187842_m1 | NM_004048.2 |
| | (control gene) | | |

The oil extract of rose flower at 1 μg/mL, after 24 hours of treatment showed significant stimulation on the expression of the following target genes:
ceramide synthase (CERS3): +33%
calmodulin 3 (CALM 3): +28%
keratin 1 (KRT1): +14%
gap junction alpha-1 protein or connexin 43 (GJA1/CX43): +23%
desmocollin 3 (DESC3): +23%

The oil extract of rose flower according to the invention therefore has an effect on epidermal clock target genes, involved in lipid metabolism, the skin barrier, cell differentiation, cell communication and/or cell cohesion.

### Example 4: Effect of the Aqueous Extract of Rose Flower on Gene Expression in Normal Human Keratinocytes

An aqueous extract of rose flower at 0.08% was tested on NHK according to a protocol similar to that described in example 1.

A butylene glycol/water excipient is used as a control.

TABLE 5
List of genes present on the microfluidic card.

| | | | RefSeq GenBank |
|---|---|---|---|
| | Gene | TaqMan item no. | accession number |
| Cell | Keratin 10 | Hs00166289_m1 | NM_000421.3 |
| differentiation | KRT10 | | |
| Epidermal | Desmoglein 1 | Hs00170047_m1 | NM_001942.3 |
| cohesion | DSG1 | | |
| | Beta-2-M | Hs00187842_m1 | NM_004048.2 |
| | (control gene) | | |

The aqueous extract of rose flower at 0.08%, after 24 hours of treatment showed significant stimulation on the expression of the following target genes:
keratin 10 (KRT10): +88%
desmoglein 1 (DSG1): +29%

The aqueous extract of rose flower according to the invention therefore has an effect on epidermal clock target genes, involved in cell differentiation, and/or epidermal cohesion.

All these biological results show that the aqueous extract of rose flower and the oil extract of rose flower according to the invention stimulate, in a complementary manner, clock genes and target genes involved in lipid metabolism, the skin barrier, cell differentiation, cell communication and/or cell cohesion.

Their combined use in a cosmetic composition is therefore advantageous in that it stimulates complementary clock genes and thus activates several metabolic pathways of interest.

Non-limiting examples of formulations are described below. Percentages are weight percentages expressed by weight based on the total weight of the composition.

### Example 5: Composition in Emulsion Form

### Aqueous Phase:

| Demineralized water | qs 100% |
|---|---|
| Glycols | 20% |
| Preservatives | 0.6% |
| Chelator | 0.04% |
| Carbomer (Carbopol ® 981) | 0.3% |
| Sodium polyacrylate (Covacryl ® MV60) | 0.2% |
| Sodium hydroxide | 0.15% |
| Rose cryoextract* | 3% |

### Fat Phase:

| | |
|---|---|
| Vegetable oil, esters, silicones | 16% |
| Satin oil* | 1% |
| Antioxidant | 0.2% |
| Fragrance concentrate | 0.4% |
| Steareth-2 | 0.8% |
| Steareth-21 | 1.5% |

*as described in the Materials and Methods section above.

The composition is prepared according to the following procedure:
the gelling agents are dispersed in the aqueous phase (excluding rose cryoextract and sodium hydroxide) which is heated to 70° C.;
the fat phase (excluding fragrance concentrate, antioxidant and satin oil) is heated to 70° C.;
satin oil is added extemporaneously just before the emulsion;
the emulsion is produced by introducing the fat phase into the aqueous phase under strong stirring;
the gelling agents are neutralized by adding sodium hydroxide and the emulsion is cooled under moderate stirring with the introduction of the fragrance concentrate, the antioxidant and the rose cryoextract at low temperature.

Applying this composition to the skin of the face promotes the rhythmic process of skin cells and thus improves lipid metabolism, the skin barrier, hydration and regeneration of the skin.

### Example 6: Composition in the Form of a Solid Dispersion of Fat, of Spherical or Spheroidal Shape

### Aqueous Phase

| Demineralized water | qs 100% |
|---|---|
| Rose cryoextract* | 3.00% |
| Phenoxyethanol | 0.42% |
| Xanthan gum | 0.36% |

### Fat Phase

| | |
|---|---|
| C₁₀₋₁₈ triglycerides | 38.84% |
| Satin oil* | 1% |
| Antioxidant | 0.1% |

*as described in the Materials and Methods section above.

The composition is prepared according to the following procedure:
the wax (C₁₀₋₁₈ triglycerides=Gatefossé Lipocire) is heated above its melting point (a few degrees) with the antioxidant and the oil extract of rose ‘satin oil’;
the melted fat phase is poured under stirring into water previously heated to the same temperature as the fat phase;
the whole is kept under stirring by a rotating system for a few minutes until the desired droplet size is obtained;
the dispersion obtained is rapidly cooled by adding previously cooled glycol water (approximately −4° C.) to solidify the lipid spheroids;
stirring is stopped when the spheroids are solidified and then recovered from the surface or filtered;
the aqueous phase is prepared by mixing water, xanthan gum, preservative and rose cryoextract;
the spheroids are recovered from the surface and incorporated into the xanthan gel containing the rose cryoextract.

Applying this composition to the skin of the face promotes the rhythmic process of skin cells and thus improves lipid metabolism, the skin barrier, hydration and regeneration of the skin.

### Example 7: Composition in the Form of Oil Droplets Stabilized by a Polymeric Membrane on the Surface, in a Gelled Aqueous Phase

### Aqueous Phase:

| Demineralized water | qs 100% |
|---|---|
| Glycols | 20% |
| Preservatives | 0.6% |
| Chelator | 0.04% |
| Acrylates/C10-30 Alkyl acrylate | |
| crosspolymer (Pemulen ® TR2) | 0.3% |
| Sodium polyacrylate (Covacryl ® MV60) | 0.2% |
| Sodium hydroxide | 0.15% |
| Rose cryoextract* | 3% |

### Fat Phase:

| | |
|---|---|
| Vegetable oil, esters, silicones | 16% |
| Satin oil* | 1% |
| Antioxidant | 0.2% |
| Fragrance concentrate | 0.4% |
| Amodimethicone (Dow Corning 2-8566 Amino Fluid ®) | 0.07% |

*as described in the Materials and Methods section above.

The composition is prepared according to the following procedure:
the gelling agents are dispersed in the aqueous phase except cryoextract and sodium hydroxide,
the emulsion is prepared at room temperature by introducing the fat phase into the aqueous phase under strong stirring,
the gelling agents are neutralized by adding sodium hydroxide before introducing the rose cryoextract.

Applying this composition to the skin of the face promotes the rhythmic process of skin cells and thus improves lipid metabolism, the skin barrier, hydration and regeneration of the skin.

1. Cosmetic composition for topical application to the skin comprising, in a physiologically acceptable medium, at least an effective amount of at least one aqueous extract of rose and at least one oil extract of rose.

2. Cosmetic composition according to claim 1, characterized in that it comprises at least one aqueous phase comprising said aqueous extract of rose and at least one fat or oil phase comprising said oil extract of rose.

3. Cosmetic composition according to claim 1, characterized in that the aqueous extract of rose and the oil extract of rose are extracts of rose flower, preferably extracts of rose flower of the Evanrat or Jardin de Granville® rose variety.

4. Cosmetic composition according to claim 1, characterized in that the cosmetic composition is in the form of a dispersion of a fat or oil phase in an aqueous phase, in particular selected from the group consisting of emulsions, macroemulsions, nanoemulsions, microemulsions, pickering emulsions, solid dispersions of fat substances, or dispersions of oil droplets stabilized by a polymeric membrane.

5. Cosmetic composition according to claim 1, characterized in that the oil extract of rose comprises an extract of rose flowers in a vegetable oil, preferably sunflower oil, in particular in a weight ratio of 1:99 to 10:90 (plant extract to oil) and is present in the composition in a content ranging from 0.1% to 10%, in particular from 0.5% to 5%, and according to a particular embodiment from 1% to 2% by weight of raw material based on the total weight of said composition.

6. Cosmetic composition according to claim 1, characterized in that the aqueous extract of rose comprises an extract of rose flowers in a polar solvent, in particular in a weight ratio of 0.5:99.5 to 10:90 (plant extract to polar solvent) and is present in the composition in a content ranging from 0.1% to 10%, in particular from 0.5% to 5%, and according to a particular embodiment from 1% to 4% by weight of raw material based on the total weight of said composition.

7. Cosmetic composition according to claim 1, characterized in that it is in the form of a dispersion of oil droplets comprising the oil extract of rose of the Evanrat or Jardin de Granville® rose variety in a gelled aqueous phase comprising the aqueous extract of rose of the Evanrat or Jardin de Granville® rose variety.

8. Cosmetic process to promote the natural rhythmic process of skin cells and/or the micronutritional balance of the skin, comprising the application to the skin, in particular of the body, face and/or neck and in particular of the face and/or neck, of a cosmetic composition as defined in claim 1.

9. Non-therapeutic cosmetic use of at least an effective amount of at least one aqueous extract of rose and at least one oil extract of rose in a cosmetic composition, as an agent to promote and/or improve the natural rhythmic process of skin cells, the micronutritional balance of the skin, the lipid metabolism of the skin, the skin barrier function, the hydration and/or regeneration of the skin.

10. Non-therapeutic cosmetic use according to claim 9, wherein the effective amount of at least one aqueous extract of rose and at least one oil extract of rose stimulates the expression of epidermal clock genes and genes involved in lipid metabolism, the skin barrier, cell differentiation, cell communication and/or cell cohesion.
